(19) United States
(12) Patent Application Publication
(10) Pub. No.: US 2007/0286884 A1
(43) Pub. Date: Dec. 13, 2007

(54) IMPLANTABLE MICROBIAL CELLULOSE MATERIALS FOR HARD TISSUE REPAIR AND REGENERATION

(75) Inventors: Gonzalo Serafica, Langhorne, PA (US); Christopher Damien, Newtown, PA (US)

Correspondence Address: FOLEY AND LARDNER LLP;SUITE 500, 3000 K STREET NW, WASHINGTON, DC, 20007, US

(73) Assignee: Xylos Corporation

(21) Appl. No.: 11/761,846

(22) Filed: Jun. 12, 2007

Related U.S. Application Data
(60) Provisional application No. 60/812,962, filed on Jun. 13, 2006.

(51) Int. Cl. A61K 31/717

(52) U.S. Cl. 424/423; 514/57

(57) ABSTRACT

This invention relates to polysaccharide materials and more particularly to microbial cellulose containing materials having suitable implantation properties for repair or replacement of hard tissue. The invention also relates to the use of the implantable microbial cellulose as a bone void filler and as a carrier vehicle for active agent delivery for repair or regeneration of hard tissue.

### CROSS-REFERENCE TO RELATED APPLICATIONS

This application claims priority to U.S. Provisional Application No. 60/812,962, filed Jun. 13, 2006, the disclosure of which is incorporated herein by reference in its entirety.

### BACKGROUND OF THE INVENTION

1. Field of Invention

This invention relates to polysaccharide materials and more particularly to microbial cellulose containing materials having suitable implantation properties for repair or replacement of hard tissue. The invention also relates to the use of the implantable microbial cellulose as a bone void filler and as a carrier vehicle for active agent delivery for repair or regeneration of hard tissue.

2. Description of the Related Art

Various materials used as implantable devices in the medical industry have been well documented and can be divided into biologic, synthetic and biosynthesized. Biologic materials used as bone void fillers include autograft tissue (a patient's own tissue), allograft (tissue from another individual of the same species) and xenograft (tissue from another species). To fill bony defects autograft remains the gold standard, however, the harvest of tissue from one part of a body to be implanted into another part carries a degree of morbidity; often, there is more postoperative pain at the harvest site than at the implant site. Allograft, as described in U.S. Pat. Nos. 5,073,373; 5,290,558; 5,510,396; 6,030,635; and 6,755,863, has been used as a medical implant for a variety of indications including as a bone graft substitute. Often there is limited availability of the material and there is a risk of disease transmission from graft to host. Xenograft, which includes animal collagen, has been implanted as bone graft substitutes (U.S. Pat. No. 5,830,493) and for use in other tissue repair and replacement (U.S. Pat. Nos. 5,810,855; 6,179,872; and 6,206,931). These materials carry the risk of disease transmission from donor to host and in addition, are often cross-linked using cytotoxic chemicals to improve their mechanical strength and degradation profile.

Synthetic materials, which include polylactic acid (PLA), polyglycolic acid (PGA), and polypropylene, have long been used as surgical sutures. These synthetic materials have been fabricated into films, mesh, and more complex three dimensional structures depending on the intended applications as described in U.S. Pat. Nos. 5,441,508; 5,830,493; 6,031,148; 6,852,330; and 6,946,003.

Additional synthetic matrices consist of calcium based materials such as calcium phosphate in the form of hydroxyapatite, dicalcium phosphate or tricalcium phosphate; calcium carbonate in the form of calcite or aragonite; calcium sulfate, calcium aluminate and composites of these materials. These matrices demonstrate variable resorption rates and characteristics. Other bone graft substitute materials include bioactive glasses and glass ceramics.

These synthetic materials possess certain physical characteristics that make them suitable as an implant material. Such properties include biocompatibility, strength, and chemical stability which can be particularly important for a specific application.

However, these synthetic materials also have limitations and disadvantages such as a limited range of physical and biochemical properties, unfavorable degradation products and profiles, leaching of chemicals, difficult handling properties, and binding of proteins meant to be released. Thus, there remains a need to explore alternative materials more suitable for specific surgical applications.

Biosynthetic materials have also been used for tissue repair and augmentation. Chitosan, for instance, can be considered biosynthetic in that it is produced by living organisms, i.e. certain shellfish. This material has been suggested for use in various medical implantable applications that include scaffolds for bone and soft tissue regeneration.

Another biomaterial that has had extensive use for surgical applications is cellulose and the use of viscose or regenerated cellulose as implantable articles. Several investigators have studied tissue biocompatibility of cellulose and its derivatives (Miyamoto, T. et al., Tissue Biocompatibility of Cellulose and its derivatives. J. Biomed. Mat. Res., V. 23, 125-133 (1989)) and examined some specific applications for the material. The oxidized form of regenerated cellulose has long been used as a hemostatic agent and adhesion barrier (Dimitrijevich, S. D., et al. In vivo Degadation of Oxidized regenerated Cellulose. Carbohydrate Research, V. 198, 331-341 (1990), Dimitrijevich, S. D., et al. Biodegradation of Oxidized regenerated Cellulose Carbohydrate Research, V. 195, 247-256 (1990)) and is known to degrade much faster than the non-oxidized counterpart. A cellulose sponge studied by Martson, et al., showed excellent biocompatibility with bone and connective tissue formation during subcutaneous implantation (Martson, M., et al., Is Cellulose sponge degradable or stable as an implantation material? An in vivo subcutaneous study in rat. Biomaterials, V. 20, 1989-1995 (1999), Martson, M., et al., Connective Tissue formation in Subcutaneous Cellulose sponge Implants in rats. Eur. Surg. Res., V. 30, 419-425 (1998), Martson, M., et al., Biocompatibility of Cellulose Sponge with Bone. Eur. Surg. Res., V. 30, 426-432 (1998)). The authors surmised that cellulose material can be a viable long term stable implant. Other forms and derivatives of cellulose have also been investigated (Pajulo, O. et al. Viscose cellulose Sponge as an Implantable matrix: Changes in the structure increase production of granulation tissue. J. Biomed. Mat. Res., V. 32, 439-446 (1996), Mello, L. R., et al., Duraplasty with Biosynthetic Cellulose: An Experimental Study. Journal of Neurosurgery, V. 86, 143-150 (1997)).

However, the prior art mentions only limited applications of microbial cellulose. For example, the use of microbial cellulose in the medical industry has been described for liquid loaded pads (U.S. Pat. No. 4,588,400), skin graft or vulnerary covers (U.S. Pat. No. 5,558,861), wound dressings (U.S. Pat. No. 5,846,213) and topical applications (U.S. Pat. No. 4,912,049). These patents have focused on the use of microbial cellulose for topical applications and have not cited particular applications as an implantable material. Mello et al., described above, suggest the use of microbial cellulose as a soft tissue dura replacement, but not for bony areas. The only patent that describes the use of microbial cellulose obtained from Acetobacter xylinum as an implant is U.S. Pat. No. 6,599,518 wherein a solvent dehydrated microbially derived cellulose material can be used specifically for tissue repair materials, tissue substitutes and bulking agents for plastic and reconstructive surgical procedures. The materials described by the '518 patent differ from the instant invention in that they possess physical characteristics such as minimal elongation and high rigidity. These attributes render the implant material non-conformable and therefore not useful for particular surgical applications such as hard tissue repair or replacement. The '518 patent does not specify processing methods other than solvent dehydration at ambient pressure to produce implantable products. The salt remaining from the process and the solvent drying at ambient pressure serve to stiffen the material. This differs from the instant invention that describes either a wet paste form of microbial cellulose or drying using a method that allows the material to be moldable to fit into irregular shaped bony defects. The form of the material in '518 allows for only minimal absorption of liquid and therefore only minimal swelling. The instant invention can have a varied fiber size depending on the processing and can therefore absorb liquid, swell to fill a space or have minimal swelling, and increased conformability over the '518 material.

Prior art suggests that a bone void filler with an active agent is preferred. Damien and Parsons [Damien C. J. and Parsons J. R. Bone graft and bone graft substitutes: A review of current technology and applications J Appl. Biomat Vol 2 Pages 187-208 (1991)] describe a variety of materials for use as carriers for bone forming growth factors. These include collagen, hydroxyapatite, tricalcium phosphate, bioglasses and polymers such as polylactic acid and polyglycolic acid. They conclude that collagen may be an essential ingredient in the makeup of a carrier for optimal osteoinductive expression of osteoinductive agents. In their review there is no mention of cellulose, especially microbial cellulose and their conclusions suggest that any effective bone graft substitute with an active agent would require collagen.

Similarly, U.S. Pat. No. 5,563,124 describes an osteogenic product and process comprising calcium carbonate and a bone growth factor. Included are materials that also contain collagen, fibrin and alginate, but no mention of microbial cellulose is made.

In U.S. Pat. No. 5,558,861, the use of microbially-produced cellulose gel as a skin graft or vulnerary cover was described. The gel is in a form of a sheet of microbial cellulose which has been chemically modified and subjected to collagen treatment to enhance its ability to culture various types of cells. It did not disclose using the material as an orthopedic matrix material that can be loaded with useful agents such as BMP's. The patent also failed to disclose the use of microbial cellulose as an implantable medical device.

An amorphous non-sheeted gel of microbial cellulose is described by Serafica et al. in U.S. Patent Application 20040142019. In this application the amorphous gel is described as a wound dressing to which various agents could be added. Knowing that agents, including bone morphogenetic proteins, have demonstrated efficacy in the wound healing, this factor was included. The application does not disclose any use of the gel or gel with factors in the repair of hard tissue when implanted into the body.

Microbial cellulose for use in dura replacement is described by Damien et al. in U.S. Patent Application 20050042263. This material differs from the current invention in its application and in its composition: the dura material is in sheet form and can either be used as an onlay or sutured into place to repair soft tissue, while the material in the current invention is in a putty, gel or paste form that can be molded or packed into hard tissue defects for the repair of bone. The cellulose in the above dura application has intact fibers, while those in the current invention are either chopped or grown in a non-fibrous form. The material in the dura application has mechanical strength characteristics so as to hold a suture and/or to achieve a water-tight closure, while the current invention will not need to hold a suture and there is no required mechanical strength. The current invention should calcify and form bone, while the material in the dura application should not demonstrate any calcification at the dural site. The current application may be a composite with calcium salts, polymers, etc. and may deliver active agents; the dura application does not describe a composite or delivery of active agents.

Microbial cellulose for use in bone repair as a porous matrix combined with calcium salts is described in Hutchens et al. [U.S. Patent Application 20040096509]. As described, their material is a porous polymer matrix that requires a minimum Young's modulus of 10 GPa and a calcium salt within some of the pores. The material in the instant invention is in a different form than that envisioned by Hutchins in that it may be chopped, milled or ground into a paste, gel or putty form that is moldable to fit into various sizes and shapes of bony defects. As such the instant invention has no strength requirement and can be much lower than the 10 GPa and retain efficacy. A lower GPa is actually preferred in the instant invention in that the material is not expected to be load bearing. Also there is no necessity of having a calcium salt with the cellulose. Rather it can be implanted as processed or materials including calcium salts or bioglasses can be admixed into the amorphous form without soaking or incubation in calcium and phosphate solutions. The resulting properties of the instant invention are therefore different from the Hutchens application.

Accordingly, heretofore, there has not been provided an acceptable implantable material comprising microbial cellulose for use in hard tissue repair, regeneration or replacement applications. There remains a need for an implantable material comprising microbial cellulose that is processed differently from previously described materials. This novel processing results in an implantable material with more desirable properties and that can be used in a wider variety of bony surgical applications in the orthopaedic, dental, periodontal, and craniomaxillofacial fields. Methods of implanting microbial cellulose such as open, laparoscopic, arthroscopic, endoscopic, or percutaneous methods are also particularly desirable and attainable with a more conformable and flowable microbial cellulose containing material.

### SUMMARY OF THE INVENTION

There is provided, in accordance with one preferred embodiment of the invention, a new class of implantable materials utilizing microbial cellulose for use in medical and surgical applications of hard tissue repair, regeneration and replacement including bone void or defect filling and spine fusion.

There is provided, in accordance with another preferred embodiment of the invention, methods of implanting microbial cellulose in a wide variety of applications that utilize the desirable physical and chemical properties of microbial cellulose.

There is provided, in accordance with another preferred embodiment of the invention, a process for the preparation of these aforementioned materials that will yield the desirable properties for particular product applications.

Yet another embodiment is a method for repairing hard tissue comprising implanting a microbial cellulose composition in a subject in need thereof, wherein the microbial cellulose composition comprises at least one agent for promoting hard tissue growth, said at least one agent being released in a controlled manner by the microbial cellulose.

### DESCRIPTION OF FIGURES

FIG. 1: Release of Bovine Serum Albumin from microbial cellulose over a 72-hour period.

FIG. 2: Stiffness of cellulose paste samples with 5 and 10% cellulose with 1 minute processing time and 5% cellulose with 5 minute processing time.

FIG. 3: BSA release profiles of cellulose paste samples with 5 and 10% cellulose with 1 minute processing time and 5% cellulose with 5 minute processing time.

FIG. 4: PHMB release profiles of cellulose paste samples with 5 and 10% cellulose with 1 minute processing time, 5% cellulose with 5 minute processing time and 5% cellulose with 1 min processing time containing 10% hydroxyapatite.

FIG. 5: Absorption profiles of cellulose paste samples with 5 and 10% cellulose with 1 minute processing time and 5% cellulose with 5 minute processing time.

FIG. 6: Donation profiles of cellulose paste samples with 5 and 10% cellulose with 1 minute processing time and 5% cellulose with 5 minute processing time.

FIG. 7: Stiffness of 5% cellulose paste samples (1 minute processing time) before drying, following drying with SCD and air-drying.

FIG. 8: Table showing pH values of various paste formulations.

### DETAILED DESCRIPTION OF THE PREFERRED EMBODIMENTS

The present invention describes an implantable material comprised of microbial cellulose. The instant implantable material has properties necessary for in vivo applications. For example, the implantable material of the instant invention is a paste that can be molded into a three-dimensional shape and possesses desired conformability characteristics.

The implantable materials of the instant invention are comprised of microbial cellulose. Those methods of preparing microbial cellulose are known to those of ordinary skill and are described, for example, in U.S. Pat. Nos. 5,846,213 and 4,912,049, which are incorporated herein by reference in their entirety. Any cellulose producing organism can be used in producing the raw biosynthetic cellulose material. However, biosynthetic cellulose produced from a static culture of Acetobacter xylinum is preferred.

The microbial cellulose content of the raw material is dependent on the amount of media supplied to the A.x. bacteria. Once the pellicle is harvested, the raw material is physically and chemically processed so as to be a suitable implantable material for medical and surgical uses. For example, the microbial cellulose is first processed and cleaned to remove all non-cellulose material embedded in the cellulose pad and then depyrogenated using chemicals such as sodium hydroxide. After depyrogenation, the cellulose may be cross-linked by irradiation or chemical means if its strength needs to be adjusted. Addition of other agents, such as glycerol, polyethylene glycol, Tween, chitosan, ethyl cellulose, hydroxypropyl cellulose or carboxymethyl cellulose can be used to modify the cellulose surface can also be performed in order to control water absorption and pliability which are desirable properties for implantable materials. The material can remain wet, moist, partially dehydrated, or totally dehydrated by air, heat, lyophilization, freeze-drying or supercritical fluid drying. The material may be further processed by chopping, grinding or milling to a paste consistency. Preferably, the processed microbial cellulose will be further sterilized for applications as medical implantable articles using standard sterilization methods such as gamma irradiation, e-beam irradiation, ethylene oxide or steam sterilization.

In one preferred embodiment, the invention provides a method for preparing an implantable device for medical and surgical applications comprising the steps of providing a microbial cellulose material and incorporating said material into an implantable device for medical and surgical applications. Once produced, the microbial cellulose may be incorporated or fashioned into medical devices by commonly known methods such as molding, cross-linking, chemical surface reaction, dehydrating and/or drying, cutting or punching. Such medical devices include tissue substitutes or scaffolds for repair or reinforcement of damaged hard tissue. For example, the instant microbial cellulose may be used as a scaffold in tissue engineering, substitution and replacement for hard tissue such as bone.

The instant microbial cellulose may be used as a substitute or scaffold in tissue engineering for orthopedic hard tissues such as bone. In this embodiment, the cellulose acts as a scaffold or trellis on which new tissue forms, orients and matures.

In a preferred embodiment, the invention provides a method of bone defect filling, consisting of an implantable composition comprising microbial cellulose and implanting said composition into a subject in need thereof. For example, the instant invention may be easily prepared as a dry or hydrated paste for direct application into bone voids. For this application the material must be conformable so as to fill the site and remain without dislocating.

In a preferred embodiment, the invention provides a method for use as an adjunct to achieve spine fusion. For this application the material must be moldable, easily implanted and remain in situ. Depending on the surgery the material can be implanted alone or as a filler for spine cage implants or cadaveric femoral ring implants.

In a preferred embodiment, the invention provides a bone graft material that is degradable. For this application the cellulose is subjected to oxidation at various levels to render it bioresorbable. Depending on the level of oxidation the material can resorb in weeks to years. This resorption can be tailored to the rate of bone formation so that voids are not created by the material degrading too quickly.

The material can be used for repairing tissue in any bony site in the skeleton including dental and periodontal applications. The microbial cellulose described can be processed using the methods described above to create a paste or gel that can be implanted to fill various sized bony defects.

The instant invention also contemplates an implantable composition comprising microbial cellulose and a medically useful agent. Any number of medically useful agents for tissue repair can be used in the invention by adding the substances to an implantable composition comprising the microbial cellulose carrier, either at any step in the manufacturing process or directly to the final composition. A medically useful agent is one having therapeutic, healing, curative, restorative or medicinal properties. Such medically useful agents include collagen and insoluble collagen derivatives, hydroxyapatite and soluble solids and/or liquids dissolved therein. Also included are amino acids, peptides, vitamins, co-factors for protein synthesis; hormones; endocrine tissue or tissue fragments; synthesizers; enzymes such as collagenase, peptidases, oxidases; cell scaffolds with parenchymal cells; angiogenic drugs and polymeric carriers containing such drugs; collagen lattices; biocompatible surface active agents, antigenic agents; cytoskeletal agents; cartilage fragments, living cells such as chondrocytes, bone marrow cells, mesenchymal stem cells, natural extracts, tissue transplants, bioadhesives, transforming growth factor (TGF-beta) and associated family proteins (bone morphogenetic protein (BMP), growth and differentiation factors (GDF) etc.), fibroblast growth factor (FGF), insulin-like growth factor (IGF-1) and other growth factors; growth hormones such as somatotropin; bone digesters; antitumor agents; fibronectin; cellular attractants and attachment agents; immuno-suppressants; permeation enhancers; and peptides, such as growth releasing factor, P-15 and the like.

The drug can be in its free base or acid form, or in the form of salts, esters, or any other pharmacologically acceptable derivatives, enantomerically pure forms, tautomers or as components of molecular complexes. The amount of drug to be incorporated in the composition varies depending on the particular drug, the desired therapeutic effect, and the time span for which the device is to provide therapy. Generally, for purposes of the invention, the amount of drug in the system can vary from about 0.0001% to as much as 60%.

The active agent may be used to reduce inflammation, increase cell attachment, recruit cells, and/or cause differentiation of the cells to repair the damaged tissue. In addition, implantable materials using microbial cellulose may be applied in a number of other useful areas, including, but not limited to other hard tissue substitutes or scaffolds.

Other objects, features and advantages of the present invention will become apparent from the following examples. It should be understood, however, that the detailed description and the specific examples, while indicating preferred embodiments of the invention, are given by way of illustration only, since various changes and modifications within the spirit and scope of the invention will become apparent to those skilled in the art from this detailed description. The invention, thus generally described, will be understood more readily by reference to the following examples, which are provided by way of illustration and are not intended to be limiting of the present invention.

### EXAMPLE 1

### Implantable Cellulose Preparation

To prepare the microbial cellulose of the invention, Acetobacter xylinum microorganisms are cultured in a bioreactor containing a liquid nutrient medium at 30 degrees Celsius at an initial pH of 3-6. The medium is based on sucrose or other carbohydrates.

The bioreactor is composed of a plastic box fitted with an airtight cover. Dimensions of the bioreactor measured 9 in×13 in. Aeration ports are made in the bioreactor that allows the proper oxygen level to be achieved.

The fermentation process under static conditions is allowed to progress for a period of about 10-14 days, during which the bacteria in the culture medium produce an intact cellulose pellicle. Once the media is expended, the fermentation is stopped and the pellicle removed from the bioreactor.

1. Processing and Depyrogenation Procedures

The excess medium contained in the pellicle is removed by mechanical compression prior to chemical cleaning and subsequent processing of the pellicle. The cellulose pellicle is subjected to a series of chemical wash steps to convert the raw cellulose film into a medical grade and non-pyrogenic implantable material. Processing starts with an 8% sodium hydroxide solution at 70-75 degrees Celsius for 1 hour, followed by a rinse in deionized water and then a soak in 0.25% hydrogen peroxide at 70-75 degrees Celsius for 1 hour.

The resulting films can be tested for pyrogens and mechanical properties. The amount of cellular debris left in the cellulose pad after processing is measured by validated Limulus Amoebocyte Lysate (LAL) testing as outlined by the U.S. Food and Drug Administration (FDA) in 21 CFR10.90. The instant cleaning process outlined above provides a nonpyrogenic cellulose pad (≦0.50 EU/ml). The steps of the LAL test are defined by the test kit manufacturer and can simply be followed to yield the pyrogen level in the cellulose film.

2. Final Product Processing

Once cleaned, the pellicles are mechanically pressed to reduce the water content. The resulting pad is cut to shape and packaged.

### EXAMPLE 2

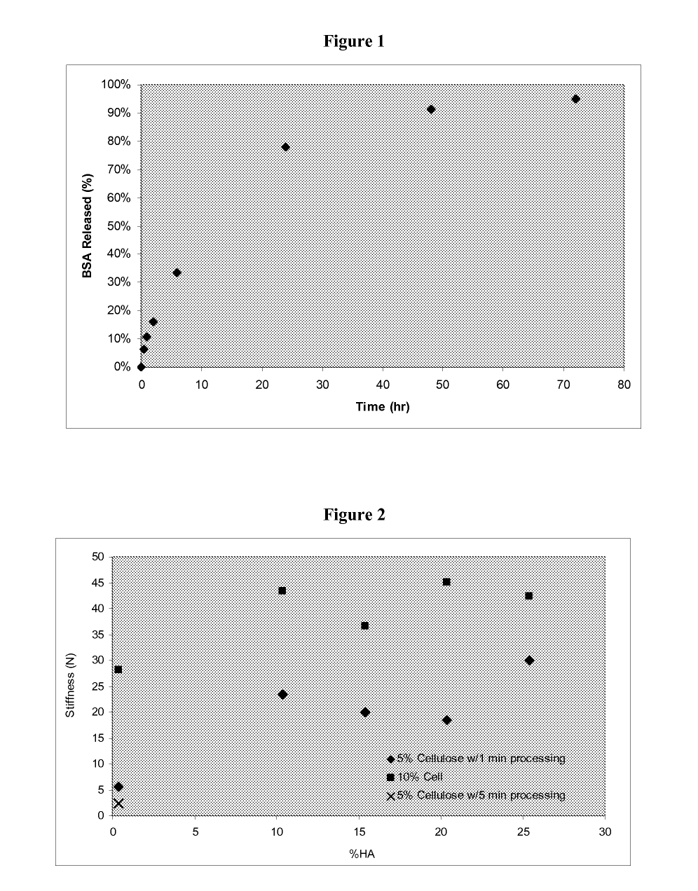

Material can be prepared the same as in Example 1, with the added step of soaking in a 1% solution of bovine serum albumin (BSA) for 24 hours. Following saturation in the BSA solution the sample is placed in a 0.9% saline solution of 20× the sample mass. Aliquots are removed from the solution at various time points to determine the BSA release profile. The BSA concentration is assessed via ultraviolet/visible spectrophotometry. FIG. 1 shows BSA was completely recovered following 72 hours in the saline solution indicating little to no binding of BSA to the cellulose.

### EXAMPLE 3

Material can be prepared as per Example 1, however prior to packaging the material can be placed in a Waring Blender with additional water to form a paste. Once processed the material is dehydrated by straining and then packaged. The fiber size can be controlled by blending time to result in materials with different physical properties. Samples containing 5% cellulose were made with processing times of one and five minutes. Stiffness testing was performed with a UNITED Tensile Tester using the circular bend procedure (ASTM Test Method D 4032). Samples were formed into discs with a diameter of between 3 and 4 cm with a thickness of between 5 and 7 mm. Stiffness values for one and five minutes processing were 5.6±1.7 and 2.3±0.7N, respectively, suggesting larger fiber sizes result in a more cohesive material.

### EXAMPLE 4

Material can be prepared as per Example 3, however, an additional flow enhancer may be added to result in a moldable paste. Tween, PEG, and carboxymethyl cellulose have been added to cellulose paste samples in concentrations ranging from 2.5 to 10% to alter the conformability characteristics of the pastes. Furthermore the addition of a flow enhancer results in reduced fluid and/or active ingredient loss during handling.

### EXAMPLE 5

A malleable paste containing 2.5% PEG 400, 10% hydroxyapatite (100-200 nm particle size), and approximately 4% cellulose.

500 milligrams of PEG 400 was mixed into 8500 milligrams of a 5% cellulose paste prepared per Example 3. After the PEG and cellulose paste components were well mixed, 1000 milligrams of 100-200 nm hydroxyapatite was mixed into the paste. The resulting paste contained a PEG 400 concentration of 2.5% (w/w), a hydroxyapatite concentration of 10% (w/w), and cellulose concentration of 4% (w/w). This provided a malleable paste with excellent formability properties and moderate water retention properties. A similar paste containing 5% PEG 400, 10% 100-200 nm hydroxyapatite, and 4% cellulose was also prepared. No differences were observed in formability or water retention properties.

### EXAMPLE 6

A malleable paste containing 5% carboxymethyl cellulose and 5% cellulose.

519 milligrams of carboxymethyl cellulose (molecular weight=250,000) was mixed into 9,574 milligrams of a 5% cellulose paste prepared per Example 3. The components were well mixed and allowed to sit for 72 hours at room temperature. This provided a malleable paste with moderate formability properties and excellent water retention properties.

### EXAMPLE 7

Material can prepared as in Example 3, however additional calcium salts (e.g. hydroxyapatite, tricalcium phosphate, calcium sulfate) can be added to form a composite paste. Paste samples with 5 and 10% cellulose were made by incorporating 10, 15, 20 and 25% hydroxyapatite (HA). Stiffness values (FIG. 2) and conformability were used to assess the handling properties of the composites. Stiffness values for composite samples with 5% cellulose increased 3-fold with the addition of 10% HA. Stiffness values remained constant with further increases in HA concentration, although conformability properties decreased. 10% cellulose samples showed a 50% increase in stiffness with 10% HA and as seen with the 5% cellulose samples the stiffness remained constant with increases in HA and similar decreases in conformability were observed.

### EXAMPLE 8

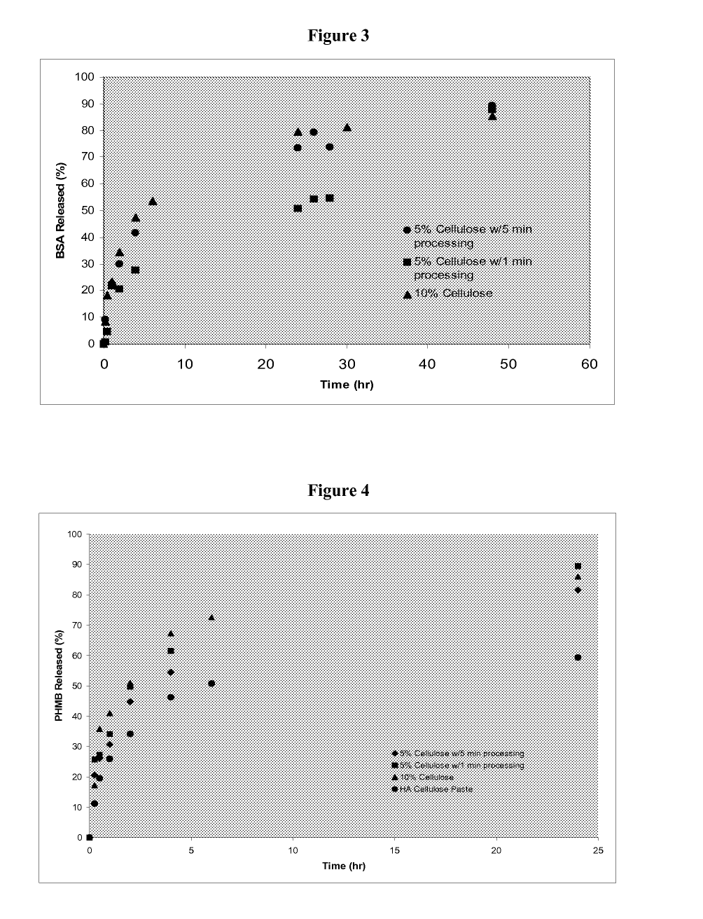

BSA release profiles were determined with materials described in Example 3. Samples were loaded by soaking in a 1% BSA solution for 24 hours. After loading the individual samples were packed into a porous nylon sample bag and then placed in an aqueous solution with a volume 20× the mass of the paste sample. Aliquots were removed at various time points and analyzed with UV/Vis spectrophotometry to determine the solution concentration which then could be used to determine the amount of BSA remaining in the paste. Measurements were taken until the solution reached the theoretical equilibrium concentration. FIG. 3 shows that the 10% cellulose sample releases BSA at a higher rate than the 5% cellulose sample reaching equilibrium at 30 hours as opposed to 48 hours for the 5% sample. Furthermore the 5% cellulose sample processed for 5 minutes shows a similar profile as the 10% cellulose sample.

### EXAMPLE 9

PHMB release profiles were determined with materials described in Example 3 and in addition a 5% cellulose sample with 10% HA was also evaluated. Samples were loaded by soaking in a 1% PHMB solution for 24 hours. After loading the individual samples were packed into a porous nylon sample bag and then placed in an aqueous solution with a volume 20× the mass of the paste sample. Aliquots were removed at various time points and analyzed with UV/Vis to determine the solution concentration which then could be used to determine the amount of PHMB remaining in the paste. Measurements were taken until the solution reached the theoretical equilibrium concentration. FIG. 4 shows that the pure cellulose samples released PHMB at similar rates reaching equilibrium at 24 hours. However, the HA containing sample still contains approximately 50% more PHMB at 24 hours indicating binding between HA and PHMB. As seen with the BSA release profiles of pure cellulose pastes, there appears to be little interaction between the cellulose fibers and PHMB allowing for complete release in a pure cellulose formulation. The addition of HA to the cellulose paste provides a means to control the release profile of the paste if there are specific interactions between additives.

### EXAMPLE 10

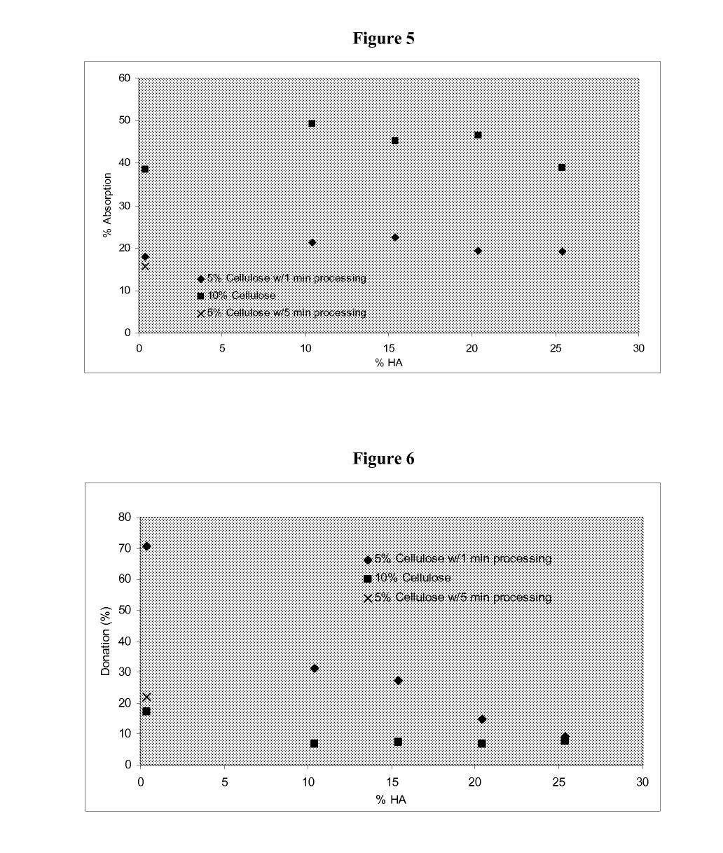

Samples were prepared as described in Example 3 and absorption and donation characteristics of the materials were determined. These samples were tested for absorption from a saturated sponge and donation to a dry surface. For the absorption test, approximately 2.5 g of the paste sample was placed on top of a sponge sitting in a 0.9% saline bath at room temperature. The liquid level was maintained at the level of the sponge. Samples were removed after 24 hr and reweighed to determine the quantity of saline absorbed by the paste, and the absorption was reported as a percentage of the initial weight of the sample. FIG. 5 shows the absorption profile for this set of pastes. As expected the absorption scales with the amount of cellulose present in the paste approximately doubling from 5 to 10% cellulose. Addition of HA results in little if any significant change in the absorption properties of the pastes at both 5 and 10% cellulose contents. Donation testing was performed by spreading between 2.5 and 3.5 g of paste over a circular area on a 3 in×3 in piece of pre-weighed smooth leather. Samples were removed after 2 hr and the leather was reweighed to determine the quantity of moisture donated to the dry surface. Donation results were reported as a percentage of the initial weight of the sample, and are shown graphically in FIG. 6. Donation decreased significantly upon increasing cellulose content to 10% from 5% cellulose. The addition of HA also affected the donation characteristics of the paste most pronounced in the 5% cellulose samples. Donation decreased approximately linearly with the additions of HA to the 5% cellulose samples resulting in an overall 80% decrease with the addition of 25% HA. Samples with 10% cellulose showed a 40% decrease irregardless of HA concentration.

### EXAMPLE 11

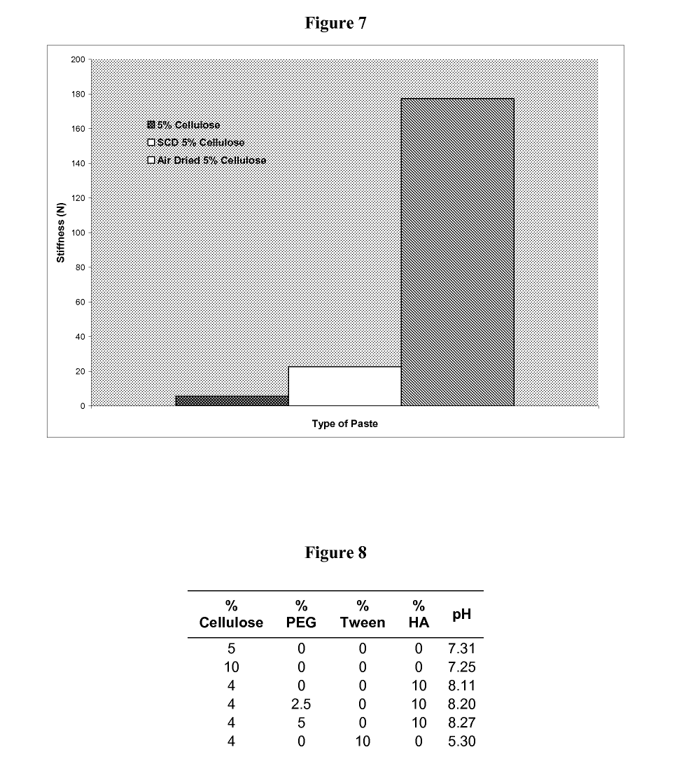

Material can be prepared as described in Example 3 and dried under ambient conditions at elevated temperatures. After drying the resulting material is hard, brittle, and non-conforming. The stiffness of the air-dried material was determined and a 30-fold increase was observed compared to the stiffness of its non-dried counterpart, as shown in FIG. 7

### EXAMPLE 12

Material can be prepared as described in Example 3 and dried using a supercritical drying (SCD) process with CO₂. The material first undergoes a solvent exchange process with methanol followed by the SCD processing. Following the SCD process the material is soft and spongy with a 3-fold increase in stiffness, with respect to the wet paste, illustrated in FIG. 7.

### EXAMPLE 13

Cellulose pastes comprised of 5% and 10% cellulose were made per Example 3. Samples containing PEG 400, Tween 80, and hydroxyapatite were prepared using the 5% cellulose paste. The pH of each sample was measured and is shown in FIG. 8.

### EXAMPLE 14

Cellulose paste materials with varying degrees of oxidation were produced with materials made per Example 1. The cellulose films were incubated in solutions of varying sodium periodate to cellulose ratios including 0.8:1, 2:1, and 4:1. Incubation was conducted in closed reaction vessels at 30 degrees Celsius within a darkened incubator for approximately 17.5 hrs. Following incubation, the samples were placed in deionized water to extract unreacted sodium periodate. The oxidized cellulose pads were then subjected to a solvent exchange process with methanol, ground in a blender, and dried using the SCD process.

### EXAMPLE 15

Oxidized cellulose paste samples containing 3% cellulose were made with material as described in Example 3. The cellulose pastes were incubated in solutions of varying sodium periodate to cellulose ratios including 0.8:1, 1.5:1, 2:1, and 4:1. Incubation was conducted in closed reaction vessels at 30 degrees Celsius within a darkened incubator for 16.5 to 17.5 hrs. Following incubation, the samples were placed in deionized water to extract unreacted sodium periodate. The oxidized cellulose pastes were then subjected to a solvent exchange process with methanol followed by SCD processing.

What is claimed is:

1. A method for repairing hard tissue comprising implanting a microbial cellulose composition in a subject in need thereof, wherein the microbial cellulose composition comprises at least one agent for promoting hard tissue growth, said at least one agent being released in a controlled manner by the microbial cellulose.

2. The method according to claim 1, wherein the microbial cellulose is produced from Acetobacter xylinum.

3. The method according to claim 1, wherein the microbial cellulose content of the composition is 1 mg/cm² to 50 mg/cm².

4. The method according to claim 1, wherein the microbial cellulose is in a hydrated state.

5. The method according to claim 1, wherein the microbial cellulose is dehydrated.

6. The method according to claim 5, wherein the microbial cellulose is dried using super critical fluid drying.

7. The method according to claim 6, wherein the supercritical fluid is carbon dioxide.

8. The method according to claim 5, wherein the microbial cellulose is dried under ambient pressure at elevated temperatures.

9. The method according to claim 1, wherein the microbial cellulose is processed by grinding, milling, chopping, or oxidation.

10. The method according to claim 5, wherein the dried cellulose is rehydrated.

11. The method according to claim 10, wherein the dried cellulose is rehydrated with a solution containing an active agent.

10. The method according to claim 1, wherein the composition is a tissue scaffold.

11. The method according to claim 1, wherein the composition is used in bone void filling.

12. A method according to claim 1, wherein at least one agent is a calcium salt.

13. A method according to claim 1, wherein at least one agent is a polymer.

14. A method according to claim 1, wherein at least one agent is a protein.

15. A method according to claim 1, wherein the cellulose is oxidized using sodium m-periodate or nitrogen dioxide

16. An implantable composition comprising an agent for promoting hard tissue growth and microbial cellulose.

17. The device according to claim 15, wherein the agent is a protein.

18. The device according to claim 16, wherein the protein is a growth factor.

19. The device according to claim 16, wherein the agent is a drug.

20. A device according to claim 16, wherein the microbial cellulose is sterilized.

21. A device according to claim 19, wherein the microbial cellulose is sterilized by ionizing irradiation.

22. A device according to claim 19, wherein the microbial cellulose is sterilized by steam and pressure.

23. A device according to claim 16, wherein the microbial cellulose is dehydrated and then sterilized by ethylene oxide.
